(19) United States
(12) Patent Application Publication
(10) Pub. No.: US 2013/0006096 A1
(43) Pub. Date: Jan. 3, 2013

(54) SYSTEM AND METHOD FOR ACCELERATED FOCUSED ULTRASOUND IMAGING

(76) Inventor: Bruno Madore, Brookline, MA (US)

(21) Appl. No.: 12/518,253

(22) PCT Filed: Dec. 10, 2007
(86) PCT No.: PCT/US07/25332

§ 371 Date: Oct. 14, 2010

Related U.S. Application Data
(60) Provisional application No. 60/869,260, filed on Dec. 8, 2006.

(51) Int. Cl. A61B 5/055

(52) U.S. Cl. 600/413

(57) ABSTRACT

Embodiments of the present invention relate to systems and methods for magnetic resonance imaging (MRI) and, more particularly, to cardiac cine MRI in which the cardiac sequences are gated retrospectively. In some embodiments, UNFOLD or related temporally- based imaging (e.g., UNFOLD-SENSE) is combined with retrospective gating to produce, for example, better images of the heart in the late diastolic part of the cardiac cycle.

### CROSS-REFERENCE TO RELATED APPLICATION

The present application claims priority to U.S. Provisional Patent Application No. 60/869,260, filed Dec. 8, 2006, which is hereby incorporated by reference herein in its entirety.

### FIELD OF THE INVENTION

Some embodiments of the present invention relate to magnetic resonance imaging (MRI) and, more particularly, to cardiac cine MRI in which the cardiac sequences are gated retrospectively.

### BACKGROUND OF THE INVENTION

Prospective gating and retrospective gating represent two different ways of reconstructing cardiac-phase images, in ECG-gated cardiac cine MRI [1-3]. While the two approaches may be identical at the acquisition stage, they differ in the way data get mapped into cardiac phases, at the reconstruction stage. Over the years, retrospective gating has become widespread, and prospective gating applications have become rare. The success of retrospective gating over prospective gating comes from its superior ability to depict the end-diastolic part of the cardiac cycle, i.e., the period shortly before an R-wave occurs. This difference between the two approaches becomes increasingly clear as the amount and/or severity of arrhythmia increases.

Cardiac cine imaging has proved to be an important test bed for methods aimed at accelerating data acquisition. Because cardiac cine is a dynamic application, in the sense that many different time frames are reconstructed, the available time axis should be utilized as part of the acceleration process. The “UNaliasing by Fourier-encoding the Overlaps using the temporaL Dimension” (UNFOLD) [4] method proposed a framework for accelerating data acquisition based on the spatiotemporal characteristics of a given imaged object. Additional details regarding UNFOLD are provided in Madore U.S. Pat. No. 6,144,873, which is hereby incorporated by reference herein in its entirety. The UNFOLD framework has been adopted and modified in a number of ways by several authors, leading to hybrid/related methods such as temporal sensitivity encoding (TSENSE) [5] and UNFOLD-SENSE [6, 7]. Other related methods include k-t BLAST and k-t SENSE. A rarely mentioned limitation of these methods, as applied to cardiac cine imaging, comes from the need to implement them on prospectively gated sequences, which are typically less popular than retrospectively gated ones. The reconstruction strategy for retrospective gating is usually more complicated than that for prospective gating, and involves a k_{y}-dependent temporal interpolation step typically believed to be incompatible with the temporal shift/rotation strategy used in UNFOLD, and in related methods.

In view of the foregoing, it would be desirable to provide systems and methods capable of implementing UNFOLD, and other imaging methods, in connection with retrospective gating of cardiac sequences.

### SUMMARY OF THE INVENTION

Some embodiments of the present invention relate to systems and methods for magnetic resonance imaging (MRI) in which cardiac sequences are gated retrospectively. For example, in some embodiments, UNFOLD or related temporally-based imaging (e.g.,

UNFOLD-SENSE) is combined with retrospective gating to produce better images of the heart in the late diastolic part of the cardiac cycle.

In some embodiments, a method is provided for accelerated cardiac cine MR imaging. Data is acquired at a first cardiac phase and a first k-space location, and at a second cardiac phase and a second k-space location. A first temporal filter is applied at the first k-space location. A second temporal filter, different from the first filter, is applied at the second k-space location. In some embodiments, the method further includes performing temporal interpolation after the temporal filtering operation, to generate data at a set of desired cardiac phases. In some embodiments, parallel imaging is also used.

In some embodiments, systems and methods for imaging an object are provided, in which k-space data about the object is transformed into a temporal frequency domain to produce temporal frequency data. The temporal frequency data is filtered (e.g., by a Fermi filter) to produce filtered data. The filtered data is transformed to either a temporal or spacial domain to produce temporal or spatial data, respectively. The temporal or spatial data is mapped to phases of movement of the object to produce mapped data. The mapped data is interpolated to produce interpolated data. At least one k-space matrix is assembled based at least in part on the interpolated data, and an image is produced from the at least one k-space matrix.

In some embodiments, raw k-space data about the object may be acquired according to a sampling function. For example, the sampling function may shift or rotate by a fixed increment from one acquisition period to a next acquisition period.

In some embodiments, at least one synthetic frame may be added to the raw k-space data to produce the k-space data for further processing. For example, in some embodiments, as many as n−1 synthetic frames may be added to the raw k-space data as required to make the total number of frames a multiple of n, wherein n is an acceleration factor of the imaging.

In some instances, the raw k-space data may be missing at least one data point. Accordingly, in some embodiments, acquiring the k-space data for further processing may include filling in the missing data point(s).

In some embodiments, the temporal or spatial data may be mapped to phases of the cardiac cycle. For example, the data may be distributed uniformly according to the phases of the cardiac cycle. As another example, only the data for the diastolic part of the cardiac cycle may be redistributed.

In some embodiments, k-space matrices may be Fourier transformed to the object domain to produce images of the object.

### BRIEF DESCRIPTION OF THE DRAWINGS

For a better understanding of the present invention, including the various objects and advantages thereof, reference is made to the following detailed description, taken in conjunction with the accompanying illustrative drawings, in which:

FIG. 1a illustrates prospective gating in cardiac cine imaging, according to which any given k-space line is acquired at multiple time points during a cardiac cycle, and the time points are directly mapped, or binned, into cardiac phases;

FIG. 1b illustrates retrospective gating in cardiac cine imaging, according to which a temporal interpolation operation is performed as time points are converted into cardiac phases, and in which time samples are distributed uniformly (as shown), or non-uniformly along the cardiac-phase axis;

FIG. 2a illustrates a conventional approach for implementing UNFOLD in connection with prospective cardiac gating, in which a regular, simplistic k_{y}-t matrix is built and the UNFOLD sampling function is shifted from frame to frame;

FIG. 2b illustrates the k_{y}-t matrix for retrospective gating, in which the acquired data is distributed along a cardiac-phase axis and much of the simplicity seen in FIG. 2a disappears;

FIGS. 3a-f illustrate a processing method for implementing UNFOLD and other UNFOLD-like methods (e.g., UNFOLD-SENSE) in connection with retrospectively gated cardiac imaging, according to some embodiments of the present invention;

FIG. 4a illustrates that for acceleration factors higher than 2, additional synthetic frames may be added to the raw cardiac data, according to some embodiments of the present invention;

FIG. 4b illustrates that to extract near-DC information (e.g., to generate sensitivity maps, treat less-dynamic material, or as part of an artifact-suppression strategy), the processing may be performed with filter(s) of different bandwidth(s) than the filter shown in FIG. 3b;

FIG. 5 illustrates data representing arrhythmia, which is a heart condition that causes the duration of a cardiac cycle to vary substantially from one heartbeat to the next and that causes large variations in the number of time samples that can be collected for different k_{y} locations;

FIG. 6a-d illustrate the results of processing a simulated, retrospectively gated cardiac cine acquisition with UNFOLD, according to some embodiments of the present invention;

FIGS. 7a-d illustrate the results of processing an in vivo accelerated dataset with UNFOLD, according to some embodiments of the present invention; and

FIG. 8 illustrates images at systole and diastole of the cardiac cycle resulting from processing a cardiac cine dataset with UNFOLD-SENSE, according to some embodiments of the present invention.

### DETAILED DESCRIPTION OF THE INVENTION

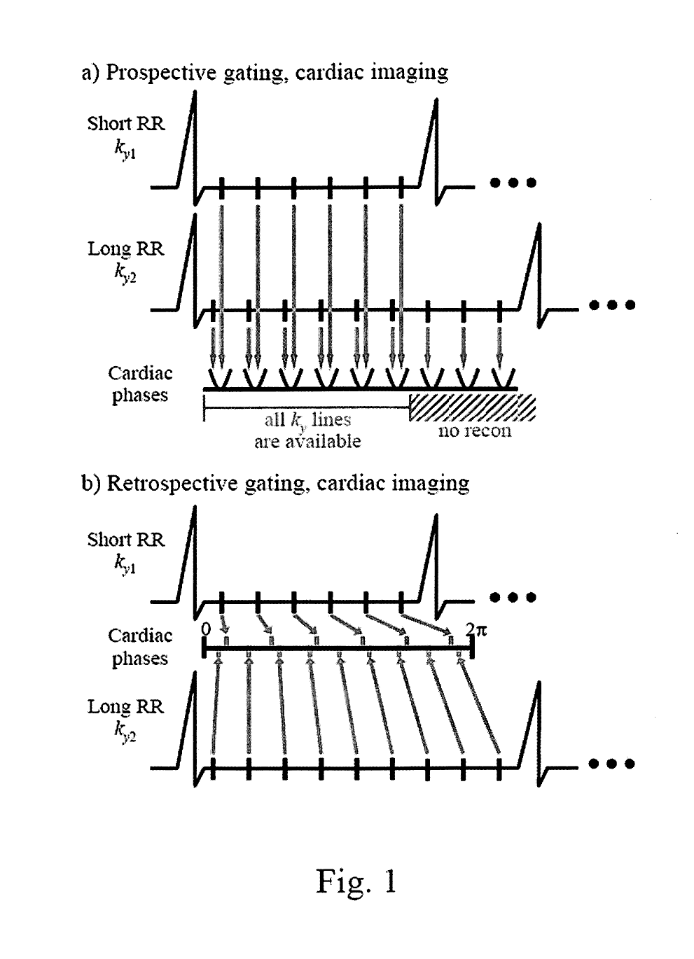

FIGS. 1a and 1b illustrate the main differences between prospective and retrospective gating. During a first heartbeat, a first set of k-space lines, which includes the line k_{y1}, gets sampled a number of times, at different cardiac phases. Each vertical black segment that intersects the electro-cardiograph (ECG) line, in-between consecutive R-waves, depicts one instance when k_{y1} gets sampled. To keep the drawing visually simple, only 6 such instances were drawn, although a higher number of about 15 to 20 time samples might be acquired, for any given k-space line, in a typical cardiac scan.

During a second heartbeat, a second set of k-space lines gets sampled, which includes the line k_{y2}. Because the second heartbeat happens in this example to be significantly longer than the first one, more samples can be acquired for k_{y2} during this second heartbeat than were acquired for k_{y1} during the first heartbeat. Up to this point, the description was concerned only with the ECG waveform and the sampling scheme, which is identical in FIG. 1a and FIG. 1b. The difference between prospective gating and retrospective gating, and thus the difference between FIGS. 1a and 1b, comes from the way data get mapped onto a cardiac phase axis, at the reconstruction stage.

As depicted in FIG. 1a, prospective gating bins k-space lines according to the order in which they were acquired. For example, the first acquisition of a given k-space line provides data toward the reconstruction of the first cardiac phase, the second acquisition of this line provides data for the second cardiac phase, and so on. In FIG. 1a, each cardiac phase can be seen as a bowl, or a bin, being filled with one copy of each k-space line (see vertical gray lines). While this strategy makes perfect sense at the beginning of the RR interval, the situation gets more complicated toward the end of the interval, during end-diastole, especially when significant arrythmia is present. As depicted in FIG. 1a, the data acquired toward the end of the longer heartbeats cannot easily be reconstructed, because the k-space locations sampled during shorter heartbeats are missing. Furthermore, the actual nature of the latest reconstructed frames is unclear, as short heartbeats may be contributing end-diastolic data, while long heartbeats may be contributing mid-diastolic data. As a consequence, prospectively gated sequences tend to have difficulties depicting accurately the end-diastolic part of the cardiac cycle.

FIG. 1b represents the strategy employed in retrospective gating. All of the acquired data for any given line gets distributed over a cardiac-phase axis ranging from 0 to 27π. Data from different heartbeats may fall at different locations along the cardiac phase axis, and for this reason, full k-space matrices cannot be readily assembled, at any cardiac phase. A temporal interpolation is required, to evaluate each one of the k-space lines at a common set of desired cardiac-phase locations. Once all k-space lines are made available through interpolation at a common set of cardiac-phase locations, these k-space lines are assembled into k-space matrices, and Fourier transformed to the object domain. From FIG. 1b, note that regardless of the length of a heartbeat, all of the acquired data is readily used in the reconstruction, and the end of the 0-2π interval does correspond to end-diastolic data, i.e., data acquired shortly before an R-wave. Both with prospective and retrospective gating, temporal interpolation is typically used to increase the number of reconstructed cardiac phases. But in prospective gating, temporal interpolation is just an optional step that could be performed at any stage of the reconstruction process, while in retrospective gating typically it must be performed at the beginning of the reconstruction process.

### UNFOLD Applied to PROSPECTIVELY GATED CARDIAC IMAGING

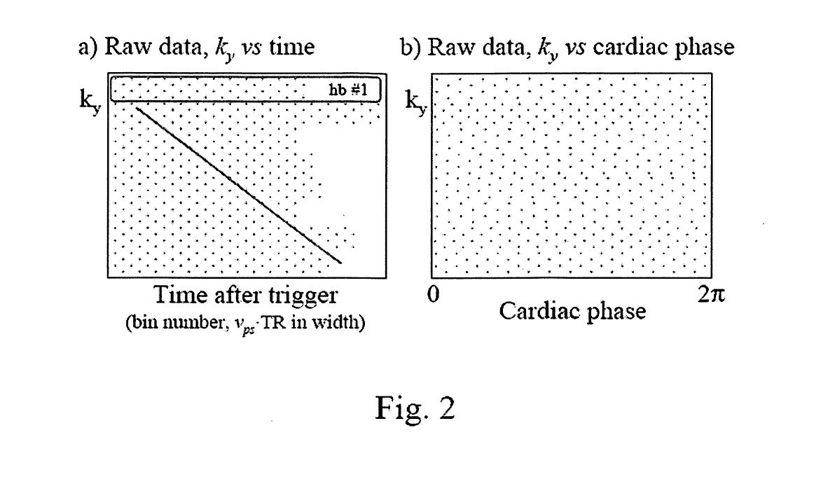

UNFOLD involves shifting or rotating the sampling function from one time point to the next, typically by a fixed increment. This sampling strategy can be represented in Xiang and Henkelman's k-t space [8], as depicted in FIG. 2a. In this example, the k-space matrix consists of only 32 lines, of which 16 are acquired at any given time frame (twofold acceleration), and the sampling function is shifted from time frame to time frame by an increment Δk_{y} equal to one k-space line. Looking at the diagonal line in FIG. 2a with slope m=Δk_{y}/Δt, the acquisition process in UNFOLD can be seen as a sheared grid in k-t space [9].

In cardiac cine imaging, vₚₛ different k-space lines get acquired in any given heartbeat, where vₚₛ stands for ‘views-per-segment’. In FIG. 2a, with 16 lines per frame and vₚₛ=2, eight heartbeats are required to complete the scan. The data acquired during the first one of these eight heartbeats is surrounded by a rectangle in FIG. 2a. Because of arrythmia, all heartbeats do not have the same duration, and accordingly not all k-space lines extend as far along the ‘time after trigger’ horizontal axis. One strategy to reduce this variation involves rejecting and reacquiring the data from heartbeats that are particularly short or long, but some degree of variation may be unavoidable, as rejecting too many heartbeats would unduly lengthen scan time.

Because k-space lines in prospective gating can readily be binned and grouped into time frames, UNFOLD can be applied here essentially in the same way as in non-gated applications. Temporal interpolation, to increase the number of reconstructed time frames, does not interfere with the UNFOLD processing, and can be performed at the end, once the UNFOLD processing is finished.

### UNFOLD Applied to Retrospectively Gated Cardiac Imaging

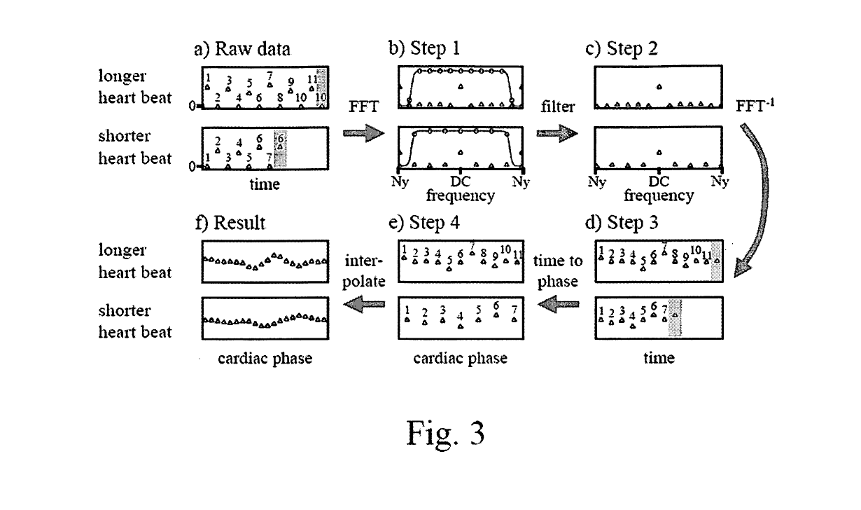

Some of the difficulties in combining UNFOLD with retrospective gating can be appreciated looking at FIG. 2b, where all of the k-space lines in FIG. 2a have been mapped to a cardiac-phase axis (as explained in FIG. 1b, for one k-space line). The nice regularity of FIG. 2a, its ‘sheared-grid’ aspect, the ability to readily apply FFTs along all dimensions, all of these simple features disappear in FIG. 2b. While the order of the various temporal and spatial operations required in an UNFOLD reconstruction can typically be permuted in a number of different ways, in the present application there is very little freedom left in the ordering of these operations. The temporal interpolation required in retrospective gating cannot be performed until UNFOLD evaluates the missing data, and FFTs to the object domain cannot be performed until the temporal interpolation has evaluated all k-space lines at a common set of cardiac-phase locations. As a consequence, typically the UNFOLD temporal processing must be performed first (on k-space points), then the temporal interpolation is performed, and finally data are transformed to the object domain. FIG. 3 illustrates how UNFOLD can be combined with retrospective gating, and the main processing steps are described in more detail below. Every data point in FIG. 3 is complex, although only the magnitude is displayed. All processing steps are illustrated both for a long and for a short heartbeat, to illustrate how the approach handles arrhythmia.

Step 1, A Temporal FFT is Applied to Each k-point, Individually (from FIGS. 3a to 3b)

All missing data points may be filled with zeros at the beginning of the processing. The data at each k-space location is Fourier transformed to the temporal frequency domain. Note that the number of time points may vary from one k-space location to another (because of arrhythmia), and accordingly the temporal FFT method may have to process arrays of different lengths for different k-space locations. For implementation with UNFOLD and/or related methods, one or more synthetic time frame(s) may have to be created before the temporal FFT is performed. This is because the FFT method interprets the first and the last time points as being connected, and continuity in the time-varying sampling scheme typically must be ensured. For example, in FIG. 3a (top plot), a given k-space location is sampled on the first and every other odd time frame, but not on the second and every other even time frame, as the sampling function was shifted for these even time frames, and some other location got sampled instead. Note that there is an alternation between sampled and non-sampled points throughout the time axis, but that the first and last (11^{th}) time points are both sampled, breaking the alternation as the first and last points get connected. To ensure continuity, the frame before last is repeated at the end, into a synthetic time frame that will be cropped away once the UNFOLD processing is completed. In this twofold acceleration example, one synthetic frame will be added every time the number of time points is odd, to make it an even number instead.

Step 2: A Temporal-Frequency Filter is Applied (from FIG. 3b to FIG. 3c)

A same temporal-frequency filter is applied to spectra obtained at all k-space locations. However, note that because different spectra may feature a different number of frequency points, the numerical values used in the actual filtering operation may differ. This point is illustrated in more detail in FIG. 3b. Both the data from a long heartbeat and a short heartbeat get filtered using a same filter, represented by a solid line. Because the temporal resolution in FIG. 3a was the same regardless of the length of the heartbeat, the Nyquist frequency, in Hz, has the same numerical value for short and long heartbeats, which justifies the use of a same filter in all cases. But as the distance between consecutive temporal-frequency points differs for long and short heartbeats, the filter gets evaluated at different frequency locations. Looking at the circles in FIG. 3b, notice that they all fall on the solid gray line of the filter, and that they are located at frequency locations where data is present. These circles represent the actual numerical values used in the filtering operation, and they differ for long and short heartbeats, as can be seen comparing the top and bottom parts of FIG. 3b.

Step 3, A Temporal FFT⁻¹ is Applied to Each K-Point, Individually (from FIGS. 3c to 3d)

Data is brought back to the time domain. Comparing the data in FIG. 3d to the raw data in FIG. 3a, note that the time points that were missing in the raw data have now been evaluated. For UNFOLD implementations where processing is performed in the spatial domain instead, this filling-in of missing k-space locations is replaced (equivalently) by a removal of aliasing artifacts. As described above, in the present application, the processing typically must be performed before k-space matrices are assembled, and is thus performed on k-space points instead of image pixels.

Step 4, Time Points Get Mapped to Cardiac Phase (from FIGS. 3d to 3e)

The synthetic frame(s), if any, are no longer needed and are cropped away. The time frames are then mapped into cardiac phases, as described in connection with FIGS. 1b and 2b. They may be distributed uniformly (as depicted here), or in any other fashion. In some embodiments, because arrhythmia results mostly from variations in the length of diastole (and not systole), only the spacing of points in the diastolic part of the cycle may get modified.

### Final Processing Steps

A temporal interpolation method interpolates the data from FIG. 3e to a common set of cardiac-phase locations, regardless of the fact that different k-space locations may have been acquired during heartbeats of different duration. Once all k-space points are available at each desired cardiac phase, a spatial FFT method produces the final result, a cardiac-phase series of images where aliasing artifacts have been suppressed.

Variations on this Method

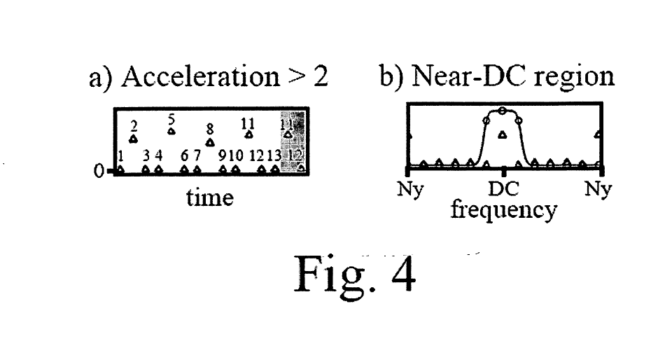

For an UNFOLD acceleration of n>2, the acquisition scheme may cycle between n different sampling patterns, and return to a given k-space location only once every n time frames. As described in FIG. 4a for n=3, as many as n−1 synthetic frames may be required, to make the number of time frames a multiple of n. In some applications, the last time frame may be very different from the first time frame, e.g., in contrast-enhanced applications where there is no contrast agent in the first frame and much enhancement in the last frame. In such applications, a larger number of synthetic frames may be required, to make the transition between last and first frames a smoother one. But in the present application, the motion is cyclical, as the heart should appear the same at phases 0 and 27π. Because of the cyclical nature of the imaged object, the simple scheme in FIGS. 3a and 4a for generating synthetic frames proves sufficient here.

When used by itself in cardiac cine imaging, UNFOLD typically assumes that one half of the FOV is less dynamic than the other half. The processing described above would be performed a first time, with the wider filter f(v) plotted in FIG. 3b, and the dynamic half would be cropped away from this first result. The processing would be repeated a second time, using the narrower (1−f(v)) filter shown in FIG. 4b, to evaluate the less dynamic half. Combining both halves, from both processing iterations, yields the final result.

Combining the Approach with Parallel Imaging

Parallel imaging is a spatial type of processing, and cannot be performed until all of the appropriate spatial frequency points or spatial pixels can be combined into a same matrix. In other words, parallel imaging typically must be performed after the temporal interpolation, which evaluates all spatial information at a common set of cardiac phases. While typically one has the choice of applying the temporal UNFOLD processing either before or after the parallel-imaging spatial processing, this choice disappears here, and UNFOLD is performed first. Except for this small difference, the extension of the present approach to methods like TSENSE or UNFOLD-SENSE will be understood by one of ordinary skill in the art based on the description set forth herein.

Object domain methods such as Cartesian SENSE would be applied after the entire processing described above, once data is in the object domain. Methods operating on k-space data, such as SMASH, would be applied at the stage shown in FIG. 3f, once all k-space points have been interpolated to a common set of cardiac phases. Methods such as TSENSE and UNFOLD-SENSE may require the UNFOLD and/or SENSE part of the processing to be performed more than once, with different settings. For example, UNFOLD can be applied by itself on the raw data, as described above, with a narrow filter (FIG. 4b), to allow sensitivity maps to be calculated [5]. A narrow filter can also be used to isolate the data to be treated with a more reliable, lower-acceleration parallel-imaging method, for artifact reduction [6].

### Examples

A simulated object was created, which consists of a rectangle (e.g., thoracic cage) containing a circle (e.g., the heart) whose radius varies according to cardiac phase. The occurrence of R-waves was randomized, to simulate the effect of arrhythmia. The proposed reconstruction method was implemented, and applied to the simulated data, to produce a cardiac-phase series of images.

Furthermore, a partially sampled in vivo dataset was simulated, by down-sampling a fully sampled one. The images were acquired on a 3T GE scanner, software release 12.0, using a product 8-element cardiac phased-array coil. Again, the number of time points available at given k_{y} locations was randomized, to simulate the effect of arrhythmia. All data processing was performed in Matlab (The MathWorks, Natick, Mass.).

### Simulated Results

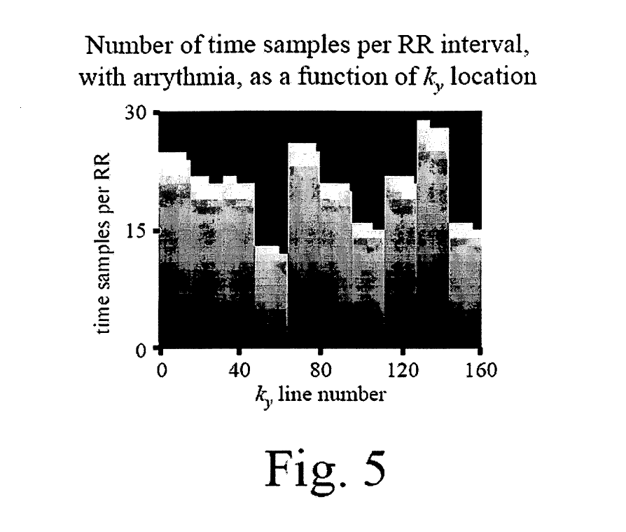

Due to arrythmia, different k-space lines are sampled more or less often, depending on the length of the particular heartbeat during which they were sampled. In this simulation, the occurrence of R-waves was randomized, with a mean RR interval of 1 s. With 16 lines sampled every heartbeat, and a TR of 3 ms, about (1000 ms /(16×3 ms))≈21 time samples could be acquired in a 1 s heartbeat. But as seen in FIG. 5, the simulated arrythmia caused large variations on the actual number of time samples obtained. A full 160-line matrix was gathered in 10 heartbeats, at a rate of 16 consecutive lines per heartbeat. Note that during any given heartbeat, the first lines are typically sampled once more than the last lines, as an R-wave often occurs before a full set could be obtained, prompting the acquisition to move on to the next set of lines. Shades of gray were used to represent cardiac phase: regardless of the actual duration of a given heartbeat, cardiac phase starts near 0 (black) at the first time sample after an R-wave, and evolves to nearly 2π (white) at the last time sample before the next R-wave.

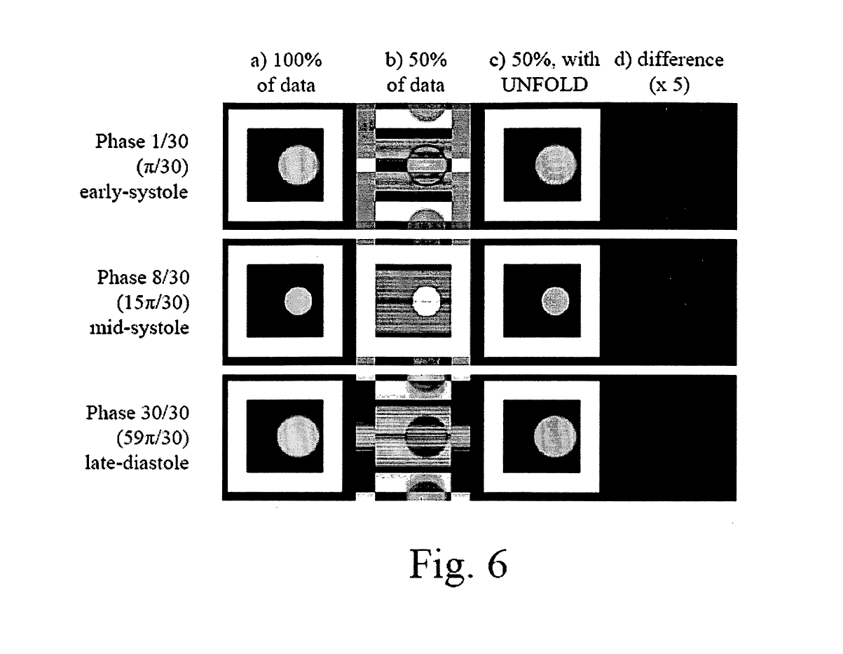

Cine images were reconstructed by applying the proposed method onto the simulated data described in FIG. 5. Thirty cardiac phases were reconstructed, ranging from a phase of π/30 (shortly after an R-wave) to a phase of 597π/30 (just before the next R-wave). Systole extended from phase 0 to π, with mid-systole at π/2. Reference images are shown in FIG. 6a, where all k-space lines were obtained. As seen in FIG. 6b, dismissing half of the k-space lines caused strong artifacts to appear. But looking at FIG. 6c, our proposed method can generate images nearly identical to the reference images, despite the fact that only 50% of the data were used. The absolute value of the difference between the images in FIG. 6a and FIG.

6c are shown in FIG. 6d, after being multiplied by 5 (these difference images would appear entirely black otherwise). In this example, UNFOLD provided 90% of the temporal bandwidth to the central half of the FOV, and 10% to the outer half. Note that using the proposed retrospectively-gated version of UNFOLD, a late-diastole frame could be reconstructed despite the presence of fairly severe arrythmia, which would be difficult using the usual prospectively-gated version of UNFOLD.

### Simulations Based on In Vivo Data

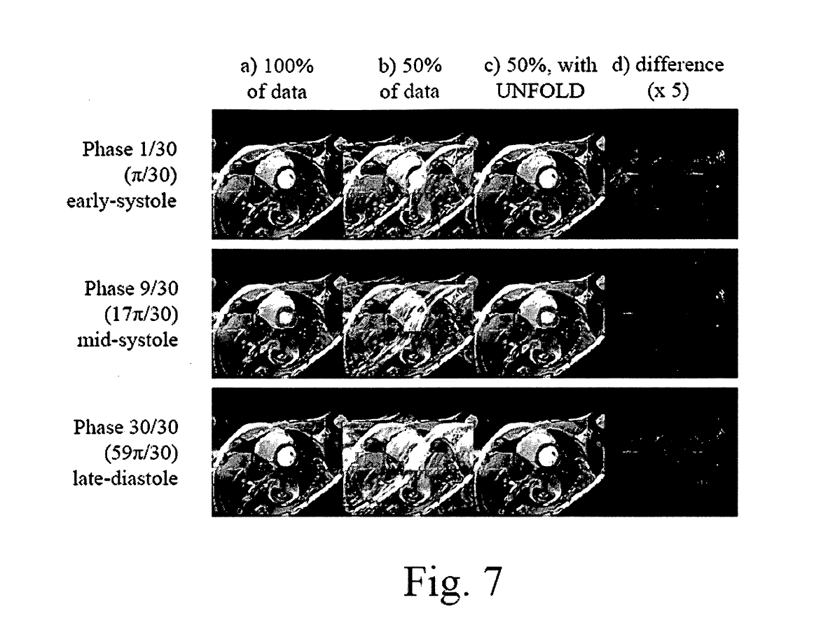

A fully sampled cardiac cine dataset was acquired. The data was interpolated in time, to simulate the presence of arrhythmia. The same heartbeat variations as in the simulated case above (see FIG. 5) were also used here. Results are shown in FIG. 7, in a format similar to FIG. 6. The difference images (FIG. 7d) were again multiplied by a factor of 5, as they would appear nearly fully black if displayed using the same windowing as in FIGS. 7a-c. Again, a late-diastole frame could be reconstructed despite the presence of fairly severe simulated arrhythmia, which would be difficult using a prospectively-gated version of UNFOLD.

### Illustrative Implementation

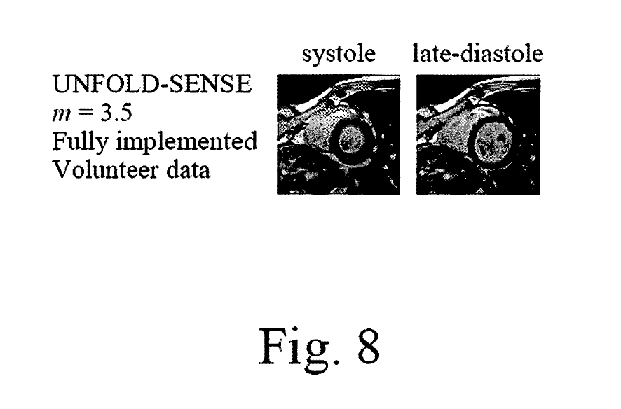

The method was fully implemented on a 3T GE scanner. A cine dataset was acquired with acceleration of 3.5 (55 lines instead of 192, including calibration lines, using a cardiac array with only 8 coil-elements), and reconstructed as described above. (Data collected from the scanner's memory in real-time, 192×192 matrix, 32×32 cm FOV, 8 mm slices, TR=3.5 ms, t res=10×TR). In a movie loop, the results play smoothly, confirming that all cardiac phases were well captured. Images at systole and end-diastole are shown in FIG. 8.

Thus, in some embodiments, the present approach allows UNFOLD and related methods such as TSENSE and UNFOLD-SENSE to be implemented on retrospectively gated cardiac sequences, which are typically preferred over prospectively-gated sequences because of their ability to better capture the end-diastolic part of the cardiac cycle. By allowing these proven methods to be implemented on the best cardiac sequences available, the present approach may significantly contribute toward improving the quality of clinical cardiac cine images.

Insofar as embodiments of the present invention described above are implementable, at least in part, using a computer system, it will be appreciated that a computer program for implementing at least part of the described methods and/or the described systems is envisaged as an aspect of the present invention. The computer system may be any suitable apparatus, system, or device. For example, the computer system may be a programmable data processing apparatus, a general purpose computer, a Digital Signal Processor, or a microprocessor. The computer program may be embodied as source code and undergo compilation for implementation on a computer, or may be embodied as object code, for example.

It is also conceivable that some or all of the functionality ascribed to the computer program or computer system aforementioned may be implemented in hardware, for example by means of one or more application specific integrated circuits.

Suitably, the computer program can be stored on a carrier medium in computer usable form, which is also envisaged as an aspect of the present invention. For example, the carrier medium may be solid-state memory, optical or magneto-optical memory such as a readable and/or writable disk for example a compact disk (CD) or a digital versatile disk (DVD), or magnetic memory such as disc or tape, and the computer system can utilize the program to configure it for operation. The computer program may also be supplied from a remote source embodied in a carrier medium such as an electronic signal, including a radio frequency carrier wave or an optical carrier wave.

Thus it is seen that cardiac cine magnetic resonance imaging with retrospective gating is provided. Although particular embodiments have been disclosed herein in detail, this has been done by way of example for purposes of illustration only, and is not intended to be limiting with respect to the scope of the appended claims, which follow. In particular, it is contemplated that various substitutions, alterations, and modifications may be made without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. The applicant reserves the right to pursue such inventions in later claims.

The following references are all hereby incorporated by reference herein in their entireties.
1. Utz J A, Herfkens R J, Heinsimer J A, Bashore T, Califf R, Glover G, Pelc N, Shimakawa A. Cine MR determination of left ventricular ejection fraction. Am J Roentgenol 1987;148:839-43.
2. Lenz G W, Haacke E M, White R D. Retrospective cardiac gating: a review of technical aspects and future directions. Magn Reson Imaging 1989;7:445-55.
3. Atkinson D J, Edelman R R. Cineangiography of the heart in a single breath hold with a segmented TurboFLASH sequence. Radiology 1991;178:357-360.
4. Madore B, Glover G H, Peic N J. Unaliasing by Fourier-encoding the overlaps using the temporal dimension (UNFOLD), applied to cardiac imaging and fMRI. Magn Reson Med 1999;42:813-828.
5. Kellman P, Epstein F H, McVeigh E R. Adaptive sensitivity encoding incorporating temporal filtering (TSENSE). Magn Reson Med 2001;45:846-852.
6. Madore B. Using UNFOLD to remove artifacts in parallel imaging and in partial-Fourier imaging. Magn Reson Med 2002;48:493-501.
7. Madore B. UNFOLD-SENSE: a parallel MRI method with self-calibration and artifact suppression. Magn Reson Med 2004;52:310-20.
8. Xiang Q S, Henkelman R M. K-space description for MR imaging of dynamic objects. Magn Reson Med 1993;29:422-8.
9. Tsao J, Boesiger P, Pruessmann K P. k-t BLAST and k-t SENSE: dynamic MRI with high frame rate exploiting spatiotemporal correlations. Magn Reson Med 2003;50:1031-42.

1. A method for accelerated cardiac cine MR imaging, the method comprising:
acquiring data at a first cardiac phase and a first k-space location; acquiring data at a second cardiac phase and a second k-space location; applying a first temporal filter at the first k-space location; and applying a second temporal filter, different from the first filter, at the second k-space location.

2. The method of claim 1, further comprising performing temporal interpolation after the temporal filtering operation, to generate data at a set of desired cardiac phases.

3. The method of claim 2, further comprising using parallel imaging.

4. A method of imaging an object, comprising: transforming k-space data about the object into a temporal frequency domain to produce temporal frequency data; filtering the temporal frequency data to produce filtered data; transforming the filtered data to a temporal or spacial domain to produce temporal or spatial data; mapping the temporal or spatial data to phases of movement of the object to produce mapped data; interpolating the mapped data to produce interpolated data; assembling at least one k-space matrix based at least in part on the interpolated data; and producing an image from the at least one k-space matrix.

5. The method of claim 4, further comprising acquiring raw k-space data about the object according to a sampling function.

6. The method of claim 5, further comprising adding at least one synthetic frame to the raw k-space data to produce the k-space data.

7. The method of claim 6, wherein adding at least one synthetic frame to the raw k-space data comprises adding as many as [symbol] 1 synthetic frames to the raw k-space data as required to make the total number of frames a multiple of n, wherein n is an acceleration factor of the imaging.

8. The method of claim 5, wherein the raw k-space data comprises at least one missing data point, further comprising filling in the at least one missing data point to produce the k-space data.

9. The method of claim 5, wherein acquiring raw k-space data about the object comprises shifting or rotating the sampling function by a fixed increment from one acquisition period to a next acquisition period.

10. The method of claim 4, wherein transforming the filtered data comprises transforming the filtered data to the temporal domain.

11. The method of claim 4, wherein transforming the filtered data comprises transforming the filtered data to the spacial domain.

12. The method of claim 4, wherein the object comprises a heart and mapping the temporal or spatial data comprises mapping the temporal or spatial data to phases of the cardiac cycle.

13. The method of claim 12, wherein mapping the temporal or spatial data comprises distributing the temporal or spatial data uniformly according to the phases of the cardiac cycle.

14. The method of claim 12, wherein mapping the temporal or spatial data comprises redistributing only the temporal or spatial data for the diastolic part of the cardiac cycle.

15. The method of claim 4, wherein producing an image from the at least one k- space matrix comprises Fourier transforming the at least one k-space matrix to the object domain.

16. The method of claim 4, wherein transforming the k-space data and transforming the filtered data are performed according to UNFOLD or UNFOLD-SENSE.

17. Apparatus for imaging an object, the apparatus configured to: transform k-space data about the object into a temporal frequency domain to produce temporal frequency data; filter the temporal frequency data to produce filtered data; transform the filtered data to a temporal or spacial domain to produce temporal or spatial data;
map the temporal or spatial data to phases of movement of the object to produce mapped data; interpolate the mapped data to produce interpolated data; assemble at least one k-space matrix based at least in part on the interpolated data; and produce an image from the at least one k-space matrix.

18. The apparatus of claim 17, wherein the raw k-space data about the object is acquired according to a sampling function.

19. The apparatus of claim 18, wherein the apparatus is further configured to adding at least one synthetic frame to the raw k-space data to produce the k-space data.

20. The apparatus of claim 19, wherein the apparatus is configured to add as many as n-\ synthetic frames to the raw k-space data as required to make the total number of frames a multiple of n, wherein n is an acceleration factor of the imaging.

21. (canceled)

22. (canceled)

23. (canceled)

24. (canceled)

25. (canceled)

26. (canceled)

27. (canceled)

28. (canceled)
